(19) United States
(12) Patent Application Publication
(10) Pub. No.: US 2012/0232634 A1
(43) Pub. Date: Sep. 13, 2012

(54) Electrotherapy Electrode Device

(76) Inventor: Richard John Fisher, III, Worthington, OH (US)

(21) Appl. No.: 13/045,842

(22) Filed: Mar. 11, 2011

(51) Int. Cl. A61N 1/04; H01R 43/00

(52) U.S. Cl. 607/149; 607/152; 29/854

(57) ABSTRACT

An electrotherapy electrode device. A generally thin, flexible top layer includes an aperture. A terminal is adjacent the top layer. The terminal has a generally planar shoulder, a post projecting from the shoulder, and a generally planar contact surface opposing the shoulder. The terminal includes a magnetic material. The post of the terminal extends through the aperture of the top layer and the shoulder of the terminal contacts the top layer.

### FIELD

The present invention relates generally to medical devices, more specifically to an electrode device used in conjunction with an electrotherapy system.

### BACKGROUND

Electrotherapeutic stimulation is recognized to promote the healing of bones and other body tissues, in addition to other advantageous physiological effects. Electrotherapy treatments normally require the establishment of a localized electrical contact with an external body surface of a living subject. This contact is typically achieved by the use of electrically conductive electrodes having extended surface areas. These surface areas are placed into electrical contact with a desired portion of the external body surface, usually through an intermediate electrode gel, liquid or other preparation designed to ensure good and continuous electrical contact between the living body surface and the electrically conductive electrode surface.

The aforementioned electrodes are attached to associated electrotherapy equipment using electrical wires, a “snap-fit” type of connector or connectors usually mechanically and electrically attaching the wires to the electrodes. Snap-fit connectors comprise a male portion having a projecting, conductive post that is attached to the electrode. The post is tapered or skirted such that a distal end of the post is larger than intermediate or base portions of the post. A female portion of the connector, having a mating receptacle, is attached to the electrode wire. When coupled together, the post of the male portion is inserted into the receptacle of the female portion. An elastically deformable or biased member within the receptacle engages the tapered portion of the post, thereby selectably retaining the male and female connector portions together. Once mated, the female portion is usually rotatable about the post of the male portion without disengaging therefrom.

A disadvantage of snap-fit electrode connectors is that they require a significant amount of force to engage and disengage the male and female portions. Consequently, a patient having an electrode attached to their skin may experience some discomfort when the electrode wires are attached. Similarly, the patient may experience discomfort if the electrode wires are accidently tugged upon. In some cases, if the electrode wires are tugged with sufficient force the electrode may be dislodged from the patient's skin before the male and female connector portions disengage, causing significant discomfort and disturbing the patient's electrotherapy treatment. There is a need for an electrode connector that is quickly and easily engageable/disengageable which lessens patient discomfort. Such a connector may include the ability for the connector portions to be rotatable with respect to each other, without inadvertently disengaging.

### SUMMARY

An electrotherapy electrode device having a magnetic connector for coupling electrotherapy leads thereto is disclosed according to an embodiment of the present invention. A magnetic terminal having a projecting post and a shoulder is placed into contact with an electrically conductive element. A top layer having an aperture sized and shaped to receive the post of the terminal is joined to the carbon conductive element, the projecting post of the terminal extending through the aperture. The terminal is thus entrapped and retained between the top layer and the conductive element, thereby holding the terminal in place and in contact with the conductive element. An electrical lead usable with the electrode device includes a contact having a receptacle that is sized and shaped to receive the post of the terminal. Magnetic attraction between the contact and the terminal selectably retains the electrical lead to the electrode device. In some embodiments the terminal and the contact of the electrical lead may both be magnetic, producing magnetic fields of complementary, attractive polarity. Alternatively, either the terminal or the contact may produce a magnetic field, the other one being made of a magnetically-attractive material.

An object of the present invention is an electrotherapy electrode device. A generally thin, flexible top layer includes an aperture. A terminal is adjacent the top layer. The terminal has a generally planar shoulder, a post projecting from the shoulder, and a generally planar contact surface opposing the shoulder. The terminal includes a magnetic material. The post of the terminal extends through the aperture of the top layer and the shoulder of the terminal contacts the top layer.

Another object of the present invention is an electrotherapy electrode device comprising a generally thin, flexible top layer. The top layer further includes a pair of spaced-apart apertures. A pair of terminals are adjacent the top layer. The terminals each have a generally planar shoulder, a post projecting from the shoulder, and a generally planar contact surface opposing the shoulder. The terminals each further include a magnetic material. The posts of the terminals extend through corresponding apertures of the top layer, the shoulder of each terminal contacting the top layer.

### BRIEF DESCRIPTION OF THE DRAWINGS

Further features of the inventive embodiments will become apparent to those skilled in the art to which the embodiments relate from reading the specification and claims with reference to the accompanying drawings, in which:

FIG. 1 is an exploded view of an electrotherapy electrode device according to an embodiment of the present invention;

FIG. 2 is a perspective view of the electrode of FIG. 1;

FIG. 3 is a view in section of the electrode of FIG. 2;

FIG. 4 is an exploded view of an electrotherapy electrode device according to another embodiment of the present invention;

FIG. 5 is a perspective view of the electrode of FIG. 4; and

FIG. 6 is a view in section of the electrode of FIG. 2.

### DETAILED DESCRIPTION

In the discussion that follows, like reference numerals are used to refer to like elements and structures in the various figures.

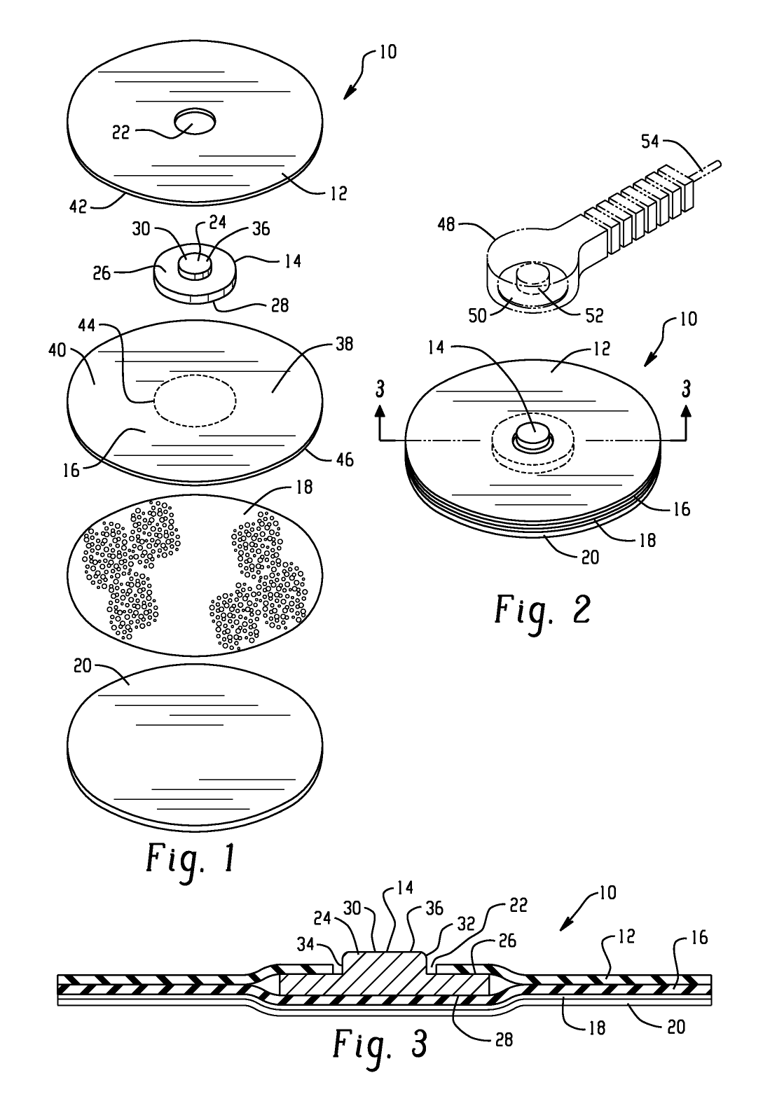

An electrotherapy electrode device 10 (hereafter “electrode 10”) is shown in FIGS. 1 through 3 according to an embodiment of the present invention. Electrode 10 comprises a top layer 12, a terminal 14, a conductive element 16, a gel layer 18 and a release liner 20.

Top layer 12 is preferably generally planar, flexible, and relatively thin. An aperture 22 extends through top layer 12. Top layer 12 and its aperture 22 may be formed using any suitable process including, but not limited to, cutting, stamping and molding. Top layer 12 may be made from any suitable material including, but not limited to, an electrically insulative material such as flexible plastic, fabric or foam.

Details of terminal 14 are shown in FIGS. 1 and 3. Terminal 14 includes a post 24 projecting from a generally planar shoulder 26, and a generally planar contact surface 28 opposing the shoulder. A distal end 30 of post 24 may include a rounded or chamfered edge 32 between a sidewall 34 and a top surface 36 of the post. Terminal 14 may be formed using any suitable process such as, without limitation, machining, casting and molding.

Terminal 14 is preferably made of a magnetic material. “Magnetic material” as used herein refers to any material that either produces a magnetic field and/or is attracted to a magnetic field. Example materials capable of producing a magnetic field include, but are not limited to, ferromagnetic and ferrimagnetic materials such as ferrites, ceramics, alnico, and rare-earth magnets such as samarium-cobalt and neodymium-iron-boron (NIB) magnets. Example materials attracted to a magnetic field include, but are not limited to, ferrous metals such as iron, nickel, cobalt, and certain steel and stainless steel alloys.

Conductive element 16 may be made of carbon, preferably a carbon fiber. The impedance of the carbon fiber is preferably low, and may approach that of a metal conductive element. Furthermore, conductive element 16 is preferably flexible, allowing the electrode 10 to conform to a body portion of the patient. In one embodiment conductive element 16 is generally planar. Conductive element 16 may be formed using any suitable process including, but not limited to, cutting, stamping and molding.

An assembly adhesive 38 is applied to an upper surface 40 of conductive element 16 to join the conductive element to a mating lower or underside surface 42 of top layer 12. Assembly adhesive 38 may be any suitable conductive or non-conductive adhesive such as, but not limited to, drying adhesives, pressure sensitive adhesives, contact adhesives, thermoplastics, and reactive adhesives. If assembly adhesive 38 is not electrically conductive a terminal clearance area 44 is provided in order to facilitate electrical contact between terminal 14 and conductive element 16 when electrode 10 is assembled.

Gel 18 may be comprised of any suitable conductive gel material. In some embodiments gel 18 is a polymeric material that is electrically conductive, preferably hydrophylic, has low surface resistivity and good adhesive properties. Gel 18 may be hypoallergenic and may optionally include a bacteriostat and/or a fungistat. Preferably, gel 18 includes adhesive properties that allow electrode 10 to be securely attached to the skin of a patient, yet allow the electrode to be removed without significant damage to the contact surface of the patient and without significant discomfort to the patient. Gel 18 may be formed using any suitable process including, but not limited to, cutting, stamping and molding. Alternatively, gel 18 may be applied to conductive element 16 in a viscous form by brushing, screening, coating or printing the gel upon the conductive element.

Release liner 20 is made of any material suitable for use with gel 18 such that the release liner is adhered to the gel until electrode 10 is to be used, and then may be easily removed without damage to the gel. Examples include, without limitation, silicone coated kraft paper and any plastic material which does not adhere excessively to gel 18. In one embodiment of the present invention release liner 20 is a layer of clear polyester plastic material. Release liner 20 may be formed using any suitable process including, but not limited to, cutting, stamping and molding.

Electrode 10 is assembled by placing terminal 14 adjacent lower surface 42 of top layer 12 such that post 24 of the terminal extends through aperture 22 and the shoulder 26 of the terminal contacts the lower surface of the top layer. Upper surface 40 of conductive element 16 is placed facially adjacent to the lower surface 42 of top layer 12 and also with contact surface 28 of terminal 14, thereby joining the conductive element to the top layer with assembly adhesive 38 therebetween and entrapping the terminal. In this state terminal 14 is in both electrical and mechanical contact with conductive element 16. Gel 18 is joined or applied to a lower surface 46 of conductive element 16, and release liner 20 is coupled to the gel.

The various components of electrode 10, particularly top layer 12, conductive element 16, gel 18 and release liner 20 may be individually formed and then assembled together. Alternatively, the components may be provided in sheet form or some other intermediate form and then cut to a desired size and shape for electrode 10 after the electrode components are assembled.

Although not mandatory in use of electrode 10, a treatment area of a patient's skin is preferably prepared by cleaning with soap and water and/or rubbing alcohol and allowed to dry. Release liner 20 of electrode 10 is removed, exposing gel 18. Electrode 10 is then placed onto the prepared treatment area of the patient's skin, the gel 18 being both facially adjacent to and in mechanical and electrical contact with the skin. The mechanical contact of gel 18 secures electrode 10 to the skin, while the electrical contact of the gel increases conductivity between the electrode and the skin. An electrical lead 48 (FIG. 2) is placed into proximity of terminal 14, the terminal being magnetically attracted to an electrical contact 50 of the electrical lead. As electrical lead 48 engages terminal 14 a receptacle 52 of the electrical contact 50 engages and receives post 24. An electrotherapy treatment signal is applied to a wire 54 of lead 48, the electrical signal being further conducted through, in turn, contact 50, terminal 14, conductive element 16 and gel 18, to the patient's skin transcutaneously.

While coupled together the magnetic attraction between contact 50 and terminal 14 resists unintentional decoupling of electrical lead 48 from the electrode 10. In the embodiment of FIGS. 1 through 3 post 24 and receptacle 52 are circularly-shaped. Accordingly, while electrical lead 48 is coupled to terminal 14 the electrical lead may be rotated about post 24 as needed without disengaging the electrical lead from the terminal. In other embodiments post 24 and receptacle 52 may have mating but non-circular shapes for various purposes, such as to prevent rotation of electrical lead 48, and orient the electrical lead with respect to electrode 10. Similarly, post 24 and receptacle 52 may have matingly keyed shapes for establishing a predetermined orientation between electrical lead 48 and electrode 10.

When it is desired to remove electrical lead 48 from electrode 10 the lead may be urged upwardly and away from the electrode with sufficient force to overcome the magnetic attraction between electrical contact 50 and terminal 14. Electrode 10 may be removed from the patient's skin by peeling the electrode away from the skin with sufficient force to overcome the adhesive properties of gel 18, much like an adhesive bandage.

In one embodiment of the present invention terminal 14 produces a magnetic field (i.e., as a “magnet”). In this embodiment contact 50 of electrical lead 48 may also produce a magnetic field having a complementary polarization such that the contact and the terminal are magnetically attracted to one another. Alternatively, terminal 14 may produce a magnetic field while contact 50 may be made of a material that does not produce a magnetic field but is attracted to a magnetic field such that the terminal and the contact are magnetically attracted to one another.

In another embodiment of the present invention terminal 14 does not produce a magnetic field, but is attracted to a magnetic field. In this embodiment contact 50 produces a magnetic field such that the contact and terminal 14 are magnetically attracted to one another.

Terminal 14 may be made from a unitary piece of material. Alternatively, terminal 14 may be made from plural pieces that are coupled or joined together. As a non-limiting example, post 24 may be made from a magnetic material and joined to a separate component made of a magnetic or non-magnetic material and comprising shoulder 26 and contact surface 28.

In the embodiment of FIGS. 1 through 3 electrode 10 is shown as generally circularly-shaped. However, other geometric shapes of electrode 10 are contemplated within the scope of the invention. Example shapes include, without limitation, rectangular, square, oval, triangular, and polygonal shapes.

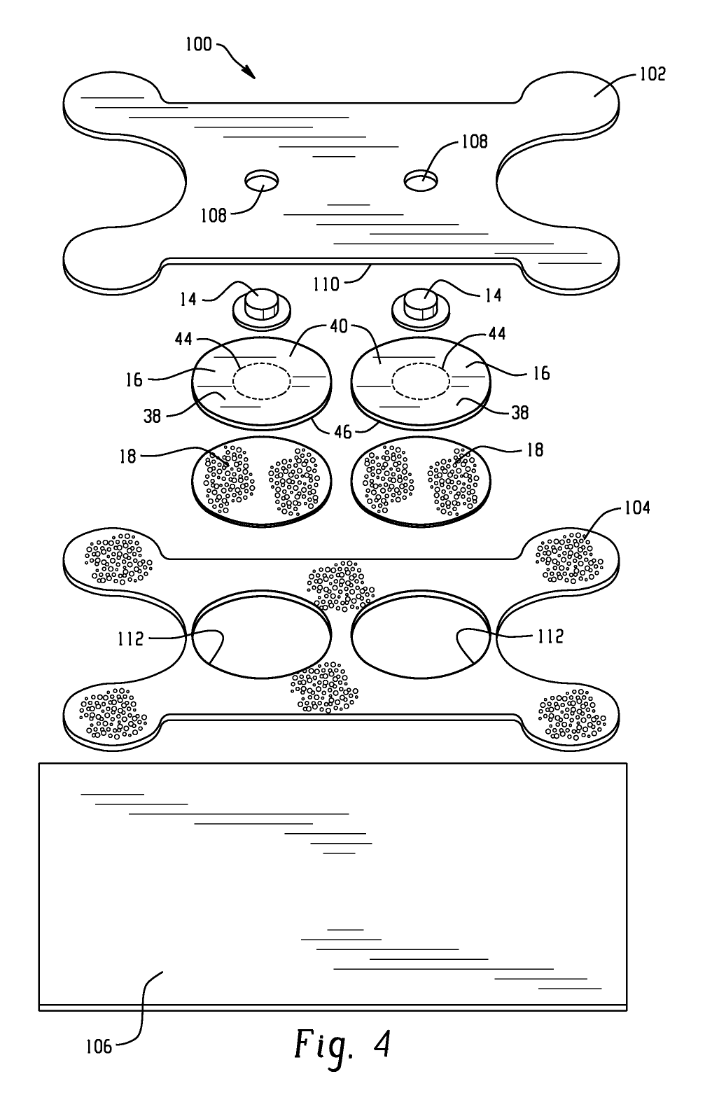

An electrotherapy electrode device 100 (hereafter “electrode 100”) is shown in FIGS. 4 through 6 according to another embodiment of the present invention. Electrode 100 includes a top layer 102, a pair of terminals 14, a pair of conductive elements 16, and a pair of areas comprising gel 18. Electrode 100 may further include a patient-attachment adhesive 104 coupled to top layer 102. A release liner 106 covers the gel 18 areas and patient-attachment adhesive 104.

Top layer 102 is generally planar, relatively thin, flexible, and is generally butterfly-shaped. A pair of spaced-apart apertures 108 extend through top layer 12. Top layer 102 is otherwise similar to top layer 12 and thus will not be detailed further.

Patient-attachment adhesive 104 may be sized and shaped to cover and conform to part or all of a lower surface 110 of top layer 102. Patient-attachment adhesive 104 may be any suitable conductive or non-conductive adhesive such as, but not limited to, drying adhesives, pressure sensitive adhesives, contact adhesives, thermoplastics, and reactive adhesives. If adhesive 104 is not electrically conductive a clearance area 112 is provided in order to facilitate electrical contact between gel 18 and the body of a patient when electrode 100 is attached to the patient.

Release liner 106 is sized and shaped to cover gel 18 and/or patient-attachment adhesive 104. Release liner 106 is otherwise similar to release liner 20 and thus will not be detailed further.

Electrode 100 is assembled by placing terminals 14 adjacent the lower surface 110 of top layer 102 such that posts 24 of the terminals extend through corresponding apertures 108 and the shoulders 26 of the terminals contact the lower surface of the top layer. Upper surfaces 40 of conductive elements 16 are placed adjacent to and into contact with the lower surface 110 of top layer 102 and with contact surfaces 28 of terminals 14, thereby joining the conductive elements to the top layer and entrapping the terminals. In this state terminals 14 are in both electrical and mechanical contact with corresponding conductive elements 16. Gel 18 is joined or applied to lower surface 46 of conductive element 16 in any manner previously described. Patient-attachment adhesive 104 is joined or applied to the lower surface 110 of top layer 102, and release liner 106 is coupled to the underside of electrode 100, covering both the exposed gel 18 and the exposed patient-attachment adhesive.

The various components of electrode 100, particularly top layer 102, conductive elements 16, gel 18, adhesive 104 and release liner 106 may be individually formed and then assembled together. Alternatively, at least some of the components may be provided in sheet form or some other intermediate form and then cut to a desired size and shape for electrode 100 after the components of the electrode are assembled.

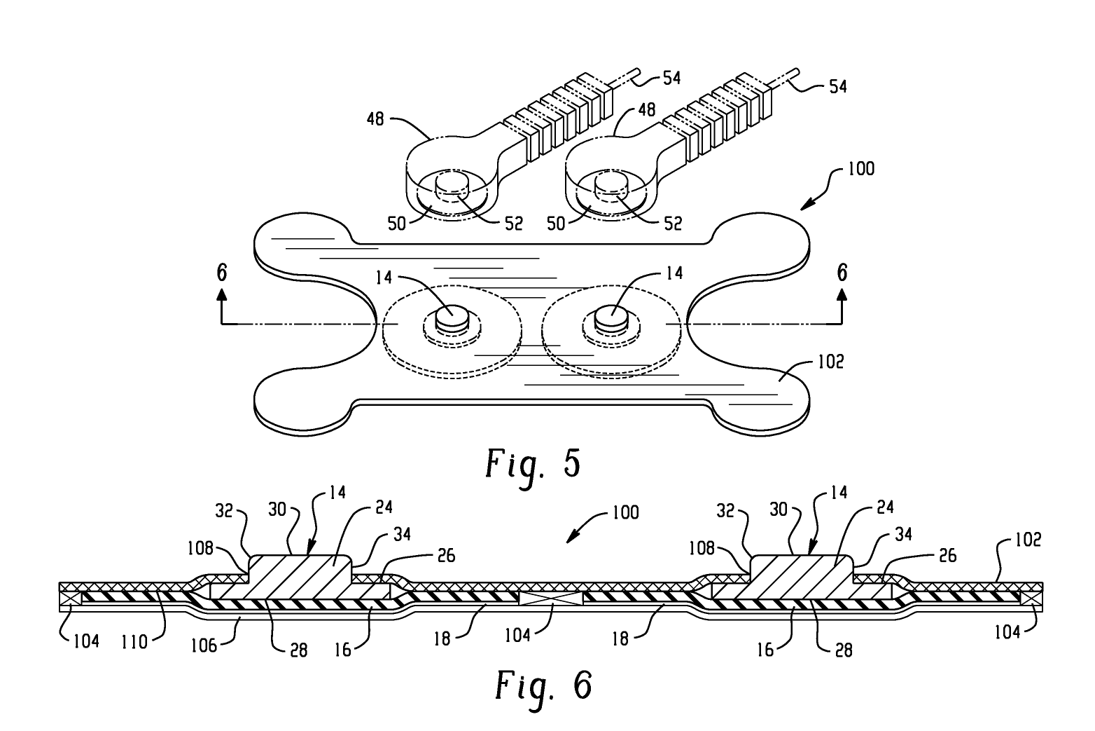

Although not mandatory, in use of electrode 100 a treatment area of a patient's skin is preferably prepared by cleaning with soap and water and/or rubbing alcohol and allowed to dry. Release liner 106 of electrode 100 is removed, exposing gel 18 and patient-attachment adhesive 104. Electrode 100 is placed onto the treatment area of the patient's skin, the gel 18 being facially adjacent the skin and in mechanical and electrical contact with the skin. The mechanical contact of gel 18 secures electrode 10 to the skin, while the electrical contact of the gel increases conductivity between the electrode and the skin. Patient-attachment adhesive 104 provides further mechanical attachment between electrode 100 and the patient's skin. A pair of electrical leads 48 (FIG. 5) are placed into proximity of terminals 14, the terminals each being magnetically attracted to a corresponding electrical contact 50 of an electrical lead. Alternatively, a unitary lead (not shown) having dual electrical contacts 50, each configured to couple to corresponding terminals 14, may be used. As each electrical lead 48 engages a terminal 14 a receptacle 52 of the electrical contacts 50 engages and receives post 24 of the terminal. An electrotherapy treatment signal is applied to a wire 54 of each lead 48, the electrical signals being further conducted through, in turn, contacts 50, terminals 14, conductive elements 16 and gel 18, to the patient's skin transcutaneously.

In the embodiment of FIGS. 4 through 6 electrode 100 is shown as generally dog-bone or butterfly-shaped. However, other geometric shapes of electrode 10 are contemplated within the scope of the invention. Example shapes include, without limitation, circular, rectangular, square, oval, triangular and polygonal shapes.

In still other embodiments of the present invention conductive element 16, which distributes the treatment signal over the treatment area, may be omitted from electrodes 10, 100. In these embodiments the size of contact surface 28 of terminal 14 may be increased to further distribute the treatment signal over the treatment area. For electrode 10, gel 18 is joined or applied to contact surface 28 of terminal 14, and release liner 20 is coupled to the gel in the absence of conductive element 16. For electrode 100, gel 18 is joined or applied to contact surfaces 28 of terminals 14, and release liner 106 is coupled to the gel and patient-attachment adhesive 104 in the absence of conductive element 16. An adhesive such as gel 18 and/or patient attachment adhesive 104 may be placed between shoulder 26 of terminal 14 and lower surfaces 42, 110 of top layers 12, 102 of electrodes 10, 100 respectively to attach the terminal to the top layer.

While this invention has been shown and described with respect to a detailed embodiment thereof, it will be understood by those skilled in the art that changes in form and detail thereof may be made without departing from the scope of the claims of the invention. For example, at least a portion of the structure and arrangement of electrodes 10, 100, including top layers 12, 102 and terminal 14, may be utilized to produce transducer elements for various medical physiological measurement applications. Examples include, without limitation, electrodes used for electrocardiogram, stress, Holter, and electromyogram tests.

What is claimed is:

1. An electrotherapy electrode device, comprising:
a generally thin, flexible top layer, the top layer further including an aperture therethrough; and
a terminal adjacent the top layer, the terminal having a generally planar shoulder, a post projecting from the shoulder, and a generally planar contact surface opposing the shoulder, the terminal including a magnetic material,
the post of the terminal extending through the aperture of the top layer and the shoulder of the terminal contacting the top layer.

2. The electrotherapy electrode device of claim 1 wherein the terminal produces a magnetic field.

3. The electrotherapy electrode device of claim 1 wherein the terminal is attracted to a magnetic field.

4. The electrotherapy electrode device of claim 1 wherein the terminal is made from a unitary piece of magnetic material.

5. The electrotherapy electrode device of claim 1 wherein the terminal is made from a plurality of pieces, at least one piece being a magnetic material.

6. The electrotherapy electrode device of claim 1, further including a flexible conductive element joined to the top layer, the terminal being entrapped between the conductive element and the top layer.

7. The electrotherapy electrode device of claim 6 wherein the conductive element is made from a flexible carbon fiber.

8. The electrotherapy electrode device of claim 6, further including a gel joined to the conductive element.

9. The electrotherapy electrode device of claim 8 wherein the gel exhibits both adhesive and electrically conductive properties.

10. The electrotherapy electrode device of claim 8, further including a release liner selectably joined to the gel.

11. An electrotherapy electrode device, comprising:
a generally thin, flexible top layer, the top layer further including an aperture therethrough;
a terminal adjacent the top layer, the terminal having a generally planar shoulder, a post projecting from the shoulder, and a generally planar contact surface opposing the shoulder, the terminal including a magnetic material, the post of the terminal extending through the aperture of the top layer and the shoulder of the terminal contacting the top layer;
a flexible conductive element joined to the top layer, the terminal being entrapped between the conductive element and the top layer;
an adhesive and electrically conductive gel joined to the conductive element; and
a release liner selectably joined to the gel.

12. An electrotherapy electrode device, comprising:
a generally thin, flexible top layer, the top layer further including a pair of spaced-apart apertures therethrough; and
a pair of terminals adjacent the top layer, the terminals each having a generally planar shoulder, a post projecting from the shoulder, and a generally planar contact surface opposing the shoulder, the terminals each further including a magnetic material,
the posts of the terminals extending through corresponding apertures of the top layer, the shoulder of each terminal contacting the top layer.

13. The electrotherapy electrode device of claim 12 wherein the terminal produces a magnetic field.

14. The electrotherapy electrode device of claim 12 wherein the terminal is attracted to a magnetic field.

15. The electrotherapy electrode device of claim 12, further including a pair of flexible conductive elements joined to the top layer, each terminal being entrapped between a corresponding conductive element and the top layer.

16. The electrotherapy electrode device of claim 15 wherein the conductive element is made from carbon fiber.

17. The electrotherapy electrode device of claim 15, further including a gel joined to each of the conductive elements.

18. The electrotherapy electrode device of claim 17 wherein the gel exhibits both adhesive and electrically conductive properties.

19. The electrotherapy electrode device of claim 17, further including a release liner selectably joined to the gel.

20. A method for producing an electrotherapy electrode device, comprising the steps of:
providing a generally thin, flexible top layer, the top layer further including an aperture therethrough;
providing a terminal having a generally planar shoulder, a post projecting from the shoulder, and a generally planar contact surface opposing the shoulder, the terminal including a magnetic material; and
placing the terminal adjacent the top layer, the post of the terminal extending through the aperture of the top layer and the shoulder of the terminal contacting the top layer.

21. An electrotherapy electrode device, comprising:
a generally thin, flexible top layer, the top layer further including an aperture therethrough;
a terminal adjacent the top layer, the terminal having a generally planar shoulder, a post projecting from the shoulder, and a generally planar contact surface opposing the shoulder, the terminal including a magnetic material, the post of the terminal extending through the aperture of the top layer and the shoulder of the terminal contacting the top layer;
an adhesive and electrically conductive gel joined to the terminal; and
a release liner selectably joined to the gel.
